(19) United States
(12) Patent Application Publication
(10) Pub. No.: US 2026/0165778 A1
(43) Pub. Date: Jun. 18, 2026

(54) APPARATUS AND METHODS FOR TRANSSEPTAL CATHETERIZATION

(71) Applicant: St. Jude Medical, Cardiology Division, Inc., St. Paul, MN (US)

(72) Inventors: Hong Cao, Maple Grove, MN (US); Troy Tegg, Elk River, MN (US); Puneet Kamal Singh Gill, Anaheim, CA (US); Salo Arias, Brooklyn Park, MN (US)

(21) Appl. No.: 19/423,388

(22) Filed: Dec. 17, 2025

Related U.S. Application Data
(60) Provisional application No. 63/735,614, filed on Dec. 18, 2024.

(51) Int. Cl. A61B 18/14; A61B 18/00; A61L 29/02; A61M 25/00

(52) U.S. Cl. CPC A61B 18/1492; A61L 29/02; A61M 25/0009; A61B 2018/00077; A61B 2018/00083; A61B 2018/00351

(57) ABSTRACT

A transseptal catheterization apparatus includes an electrically-conductive core, an energy delivery element at the distal end of the core, a coil or insulative filler surrounding at least a portion of the core, and an insulative jacket surrounding at least a portion of the core. The coil may be radially adjacent the core with the insulative jacket surrounding the coil. Alternatively, the insulative jacket may be radially adjacent the core and the coil may surround the insulative jacket. The energy delivery element may be a tip electrode conductively coupled to the core. An insulative bushing may be positioned between the tip electrode and the coil to electrically isolate the tip electrode from the coil. The exterior surface(s) of the energy delivery element and/or the coil may be dimpled or roughened.

### CROSS-REFERENCE TO RELATED APPLICATIONS

This application claims the benefit of U.S. provisional application No. 63/735,614, filed 18 Dec. 2024, which is hereby incorporated by reference as though fully set forth herein.

### FIELD

The present disclosure relates generally to apparatus and methods for penetrating tissue within the body. In particular, the instant disclosure relates to apparatus and methods for crossing the interatrial septum from the right atrium in order to access the left atrium, a procedure known and referred to herein as “transseptal catheterization.”

### BACKGROUND

The human heart includes a right ventricle, a right atrium, a left ventricle, and a left atrium. The right atrium is in fluid communication with the superior vena cava (SVC) and the inferior vena cava (IVC). The tricuspid valve separates the right atrium from the right ventricle. The right atrium is separated from the left atrium by a septum that includes a thin membrane known as the fossa ovalis.

There are several diagnostic and therapeutic procedures in which a catheter is advanced within a guide (or introducer) sheath or over a guidewire, through a subject's vasculature, and into various chambers and across valves of the heart. Certain such procedures require the catheter to be placed in the subject's left atrium.

Yet, the left atrium is the most difficult chamber of the heart to access transluminally. Access to the left atrium through the pulmonary artery is not possible, and approaches from the left ventricle are challenging.

Accordingly, one accepted method of accessing the left atrium is transseptal catheterization. In transseptal catheterization, a catheter is inserted through the femoral or left subclavian vein into the right atrium, followed by penetration of the interatrial septum, typically across the fossa ovalis, to gain entry to the left atrium.

In many cases, transseptal catheterization requires the use of multiple devices, which may be separate components or part of an integrated kit. These devices generally include an introducer sheath, a dilator, and a needle. In some instances, a guidewire is also provided over which the introducer may be inserted into the body. Similarly, the needle may include a stylet in order to enhance its pushability.

One conventional approach to transseptal catheterization, which will be familiar to those of ordinary skill in the art, involves advancing the dilator through the patient's vasculature, optionally with the aid of a guide wire and/or introducer sheath, to the fossa ovalis. Once the fossa ovalis is located, the needle can be advanced through the dilator to penetrate the interatrial septum. Next, the dilator (and/or introducer sheath, as applicable) is advanced through the resulting perforation and into the left atrium. The needle can then be removed and exchanged for a guidewire into the left atrium before further advancing the dilator and introducer sheath.

Afterwards, the dilator and guidewire can be removed altogether and a diagnostic or therapeutic medical device, such as an electrophysiology mapping catheter or an ablation catheter, can be inserted in its place and advanced into the left atrium.

Alternatively, the introducer sheath and dilator can be removed with the guidewire remaining in the left atrium. Subsequently, a new device can be advanced over the guidewire and into the left atrium.

### BRIEF SUMMARY

Disclosed herein is an apparatus for transseptal catheterization including: an electrically-conductive core having a proximal end, a distal end, and a length extending from the proximal end to the distal end; an energy delivery element positioned at the distal end of the core; a coil circumferentially surrounding a first portion of the length of the core; and an insulative jacket circumferentially surrounding a second portion of the length of the core.

The coil may be radially adjacent the core and the insulative jacket may circumferentially surround a portion of a length of the coil. The coil may include an exposed segment extending beyond a distal end of the insulative jacket. The exposed segment of the coil may have a length between about 0.10 mm (0.004 inches) and about 0.70 mm (about 0.027 inches), between about 0.20 mm (0.008 inches) and about 0.30 mm (0.012 inches), or between about 0.22 (0.009 inches) and about 0.28 mm (about 0.011 inches), such as about 0.25 mm (about 0.010 inches).

The coil may be conductively coupled to the core. For instance, the coil may be in direct conductive contact with the core.

Alternatively, the coil may be electrically isolated from the core and the energy delivery element. For instance, the insulative jacket may be radially adjacent the core and the coil may circumferentially surround a portion of a length of the insulative jacket.

The energy delivery element may be configured to deliver sufficient energy (e.g., ablative energy) to a tissue (e.g., the fossa ovalis) adjacent the energy delivery element to permit the apparatus to penetrate the tissue (referred to as “penetrating energy”).

The energy delivery element may include a tip electrode conductively coupled to the distal end of the core. Alternatively, the energy delivery element may include a solder ball on the distal end of the core. The tip electrode and/or solder ball may have dimensions to match that of the core.

Optionally, an insulative bushing may be positioned between a proximal end of the tip electrode and a distal end of the coil to electrically isolate the tip electrode from the coil.

Optionally, an insulative component may be included proximate the interface between the energy delivery element and the insulative jacket. Suitable materials for the insulative component include epoxy coating (e.g. Electrobond®), various ceramics, alumina, diamond-like carbon (DLC) coatings (e.g., CeraToughTM-D (IBC Coatings Technologies, Ltd.; Lebanon, IN)), and polyimide coatings.

The diameter of the core may taper moving distally along the length of the core. For example, the diameter of the core at the proximal end may be between about 0.25 mm (0.01 inches) and about 1.27 mm (0.05 inches), between about 0.38 mm (0.015 inches) and about 1.02 mm (0.04 inches), or between about 0.51 mm (0.02 inches) and about 0.76 mm (0.03 inches).

The diameter of the core at the distal end may be between about 0.051 mm (0.002) inches and about 0.25 mm (0.01 inches), between about 0.10 mm (0.004 inches) and about 0.20 mm (0.008 inches), or between about 0.15 mm (0.006 inches) and about 0.18 mm (0.007 inches).

The diameter of the core may taper over a length along the core of between about 12.7 mm (0.5 inches) and about 508 mm (20 inches), between about 50.8 mm (2 inches) and about 305 mm (12 inches), or between about 152 mm (6 inches) and about 254 mm (10 inches).

The core may include a metal or metal alloy, such as one or more of stainless steel, platinum, platinum-iridium, and Nitinol (nickel titanium alloy).

The coil may include a heavy metal, such as one or more of tungsten, platinum, and gold, or alloys thereof.

The insulative jacket may include a non-conductive polymer, such as one or more of polytetrafluoroethylene (PTFE), polyether ether ketone (PEEK), nylon, polyimide (PI), epoxy, polyolefin, or a polyether block amide (e.g., PEBAX). The insulative jacket may also include a coating, such as an epoxy coating (e.g. Electrobond® epoxy coating, Surface Solutions Group, LLC; Chicago, IL), polyimide coating, ceramic coating, or alumina coating. An adhesive may be used to join the insulative jacket to the core.

The energy delivery element may include one or more of platinum, platinum iridium, palladium, gold, and stainless steel.

At least one of an exterior surface of the energy delivery element and an exterior surface of the coil may include a roughened texture. The roughened texture may be formed on an exterior surface of at least a distal portion of the coil. The roughened texture may include a distribution of dimples. The roughened texture may be fabricated using laser ablation, chemical etching, electrochemical machining, spray coating, plasma spraying, or sputtering. The roughened texture may have an average roughness (Ra) between about 1 micron and about 5 microns, between about 2 microns and about 4 microns, or about 3 microns.

The energy delivery element may include a conductive plating, such as a conductive plating including at least one of platinum and gold.

The distal portion of the coil may likewise include a conductive plating, such as a conductive plating including one or more of platinum and gold.

A proximal segment of the core may be exposed beyond a proximal end of the coil and a proximal end of the insulative jacket. The exposed proximal segment of the core may be configured to be conductively connected to a power supply.

It is also contemplated to include a second coil radially adjacent the core and the coil.

Also disclosed herein is a guidewire for radiofrequency transseptal catheterization. The guidewire includes: an electrically-conductive core having a proximal end, a distal end, and a length extending from the proximal end to the distal end; a coil circumferentially surrounding a portion of the length of the core, wherein the coil is conductively coupled to the core; and an insulative jacket circumferentially surrounding a portion of a length of the coil, wherein a distal segment of the coil is exposed beyond a distal end of the insulative jacket to act as a conductive element to deliver sufficient energy to a tissue adjacent the energy delivery element to permit the guidewire to penetrate the tissue.

A proximal segment of the core may be exposed beyond a proximal end of the coil and a proximal end of the insulative jacket and may be configured to be conductively connected to a radiofrequency generator.

The guidewire may also include a tip electrode conductively coupled to at least one of the distal end of the core and the exposed distal segment of the coil.

The instant disclosure also provides a guidewire for radiofrequency transseptal catheterization including: an electrically-conductive core having a proximal end, a distal end, and a length extending from the proximal end to the distal end; a tip electrode coupled to the distal end of the core and configured to deliver sufficient energy to a tissue adjacent the tip electrode to permit the guidewire to penetrate the tissue; an insulative jacket circumferentially surrounding a portion of the length of the core; and a coil circumferentially surrounding a portion of a length of the insulative jacket, wherein the coil is electrically isolated from the core and the tip electrode.

The guidewire may also include an insulative (e.g., electrically-and/or thermally-insulative) bushing positioned between a proximal end of the tip electrode and a distal end of the coil to electrically isolate the coil from the tip electrode.

Further, a method of manufacturing an apparatus for transseptal catheterization may include the following steps: forming an electrically-conductive core having a proximal end, a distal end, and a length extending from the proximal end to the distal end; positioning an energy delivery element at the distal end of the core; forming a coil to circumferentially surround a first portion of the length of the core; and forming an insulative jacket to circumferentially surround a second portion of the length of the core.

The coil may be radially adjacent the core. The step of forming the insulative jacket to circumferentially surround the second portion of the length of the core may further include forming the insulative jacket to circumferentially surround a portion of a length of the coil or the entire length of the coil.

Alternatively, the insulative jacket may be radially adjacent the core. The step of forming the coil to circumferentially surround the first portion of the length of the core may further include forming the coil to circumferentially surround a portion of a length of the insulative jacket.

The step of positioning the energy delivery element at the distal end of the core may include securing a tip electrode to the distal end of the core. Alternatively, the step of positioning the energy delivery element at the distal end of the core may include forming a solder ball on the distal end of the core or plasma welding a metal ball from the core and/or coil.

The method may also include forming a roughened texture surface on at least one of the coil and the energy delivery element, such as by laser roughening the at least one of the coil and the energy delivery element. Other roughening methods such as chemical etching, electrochemical machining, spray coating, plasma spraying, or sputtering, are also contemplated to create the roughened texture surface.

Still further, the instant disclosure provides an apparatus for transseptal catheterization including: an electrically-conductive core having a proximal end, a distal end, and a length extending from the proximal end to the distal end; an energy delivery element positioned at the distal end of the core; an insulative filler circumferentially surrounding a first portion of the length of the core; and an insulative jacket circumferentially surrounding a second portion of the length of the core.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 illustrates representative elements of an apparatus for transseptal catheterization according to aspects of the present disclosure.

FIG. 2A illustrates a first embodiment of a radiofrequency transseptal guidewire according to the instant disclosure.

FIG. 2B illustrates a variation of the radiofrequency transseptal guidewire of FIG. 2A that incorporates an optional distal insulative coating.

FIGS. 3A and 3B illustrate a second embodiment of a radiofrequency transseptal guidewire according to the instant disclosure.

FIGS. 4A and 4B illustrate variations of a third embodiment of a radiofrequency transseptal guidewire according to the instant disclosure. In particular, FIGS. 4A and 4B illustrate a transseptal radiofrequency guidewire that uses a filler material in lieu of a coil, and FIG. 4B illustrates a variation of the radiofrequency transseptal guidewire of FIG. 4A that incorporates optional distal electrodes (e.g., ring electrodes).

FIGS. 5A and 5B depict textured surfaces of an energy delivery element or tip electrode as may be used in connection with a transseptal guidewire (e.g., as shown in FIGS. 2, 3A, 3B, 4A, and/or 4B).

FIG. 6 is a diagrammatic and block diagram view of an illustrative system such as may be used in accordance with aspects of the instant disclosure.

FIGS. 7 and 8 illustrate steps in a transseptal catheterization procedure.

FIGS. 9A and 9B are illustrative curvatures of a transseptal guidewire with a J-curve or shepherd's hook tip configuration.

FIGS. 10A and 10B are illustrative curvatures of a transseptal guidewire with a pigtail tip configuration.

While multiple embodiments are disclosed, still other embodiments of the present disclosure will become apparent to those skilled in the art from the following detailed description, which shows and describes illustrative embodiments. Accordingly, the drawings and detailed description are to be regarded as illustrative in nature and not restrictive.

### DETAILED DESCRIPTION

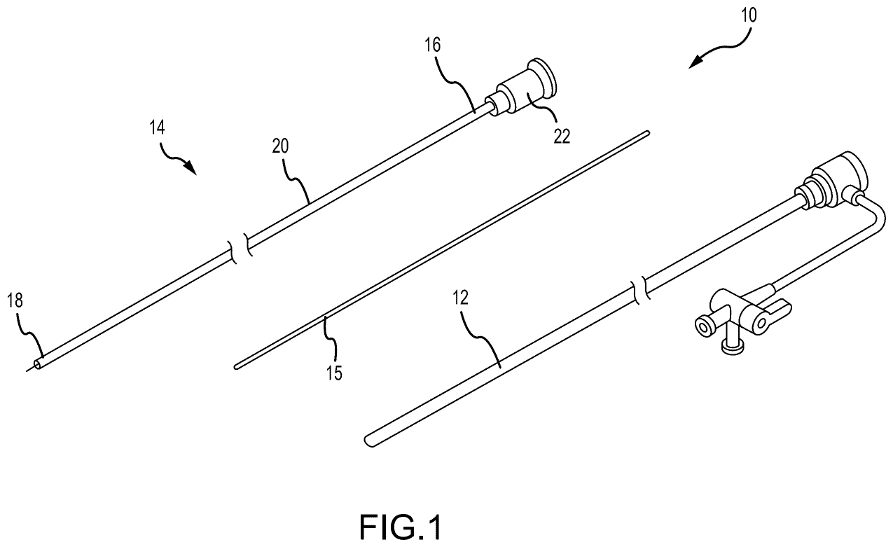

Referring now to the drawings, FIG. 1 illustrates various elements of a representative apparatus 10 for transseptal catheterization, generally including an introducer 12, a dilator 14, and a guidewire 15. Insofar as the construction of introducer 12, dilator 14, and guidewire 15 will be familiar to those of ordinary skill in the art, they will only be discussed in detail herein to the extent necessary to understand the instant disclosure. By way of example only, however, introducer 12 may be the Agilis™ N×T Steerable Introducer (Abbott Laboratories; Abbott Park, Illinois). As further examples, introducer 12 may be a Swartz™ Braided Transseptal Guiding Sheath or a Fast-Cath™ Introducer Sheath, both also of Abbott Laboratories.

As shown in FIG. 1, dilator 14 generally includes a proximal end 16, a distal end 18, and a body 20 extending therebetween. Dilator 14 can be provided in various outer diameters (i.e., French sizes), in various lengths, and/or with various preset or adjustable curvatures of distal end 18, as may be desirable for any particular application of dilator 14.

Accordingly, the specific dimensions and shapes of dilator 14 depicted and described herein are intended to be exemplary and illustrative rather than limiting. Indeed, those of ordinary skill in the art will appreciate that various modifications can be made thereto without departing from the scope of the instant disclosure. For instance, although the outer diameter of body 20 may be about 0.110 inches (sized to fit, for example, in a standard 8.5 French introducer 12), the outer diameter may be made larger or smaller depending on the intended use of dilator 14 and/or the French size of introducer 12 to be used in conjunction therewith.

Likewise, various materials suitable for use in the construction of body 20 of dilator 14 will be known to those of ordinary skill in the art. By way of illustration only, however, such materials include, without limitation, various nylon polymers (e.g., Nylon 12), polyether block amide elastomers in various durometers (e.g., PEBAX 72D (Arkema Inc.; King of Prussia, PA), high-density polyethylene (HDPE), and other thermoplastics.

It is further contemplated that body 20 may combine sections of different materials. These sections may be arranged in layers, such as by co-extrusion, and/or in abutment along the length of body 20. As but one example, the majority of body 20 may be made of Nylon 12, while about a 51 mm (about 2 inch) long segment of body 20 proximate distal end 18 may be made of PEBAX 72D. This configuration is advantageous insofar as Nylon 12 is stiffer and can improve pushability of the proximal portion of body 20, while PEBAX 72D is softer and renders the distal end of body 20 more compliant to pass through and/or be deflected by an introducer or sheath.

A dilator hub 22, which will be familiar to the ordinarily-skilled artisan, may be attached to proximal end 16 of dilator 14. Amongst other functions, dilator hub 22 can be used to deliver irrigation to distal end 18 of dilator 14 and/or for aspiration of the device. Further, a hemostasis valve adapter including a Luer lock fitting, can be attached to dilator hub 22 with guidewire 35 (described further below) inserted into dilator 14.

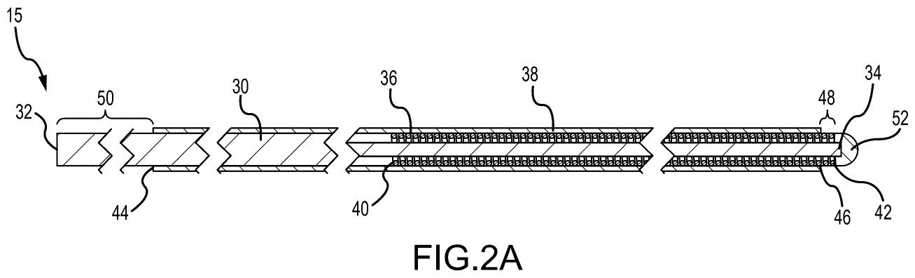

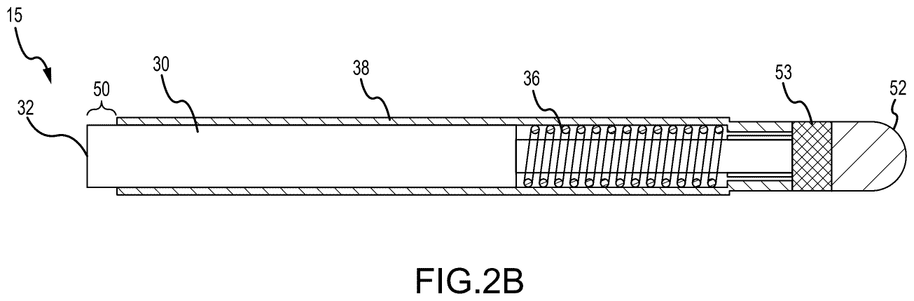

FIG. 2A illustrates guidewire 15 according to a first embodiment of the disclosure. As seen in FIG. 2A, guidewire 15 includes an elongate core 30 having a proximal end 32 and a distal end 34. Core 30 is electrically-conductive and may, for instance, be made of solid metal, such as 304 stainless steel or platinum, or a metal alloy, such as Nitinol (nickel titanium alloy) or platinum-iridium, or another suitable conductor.

Core 30 is circumferentially surrounded, as successive radially-adjacent components, by a coil 36 and an insulative jacket 38. As used herein, the term “radially-adjacent” is used to refer to radially-arranged neighboring components of guidewire 15. For instance, in the embodiment of guidewire 15 shown in FIG. 2A, coil 36 is radially-adjacent both core 30 and insulative jacket 38, while insulative jacket 38 is only radially-adjacent coil 36. The term “radially-adjacent” is not, however, limited to configurations where the radially-arranged neighboring components are in physical contact with each other, though such configurations are within the meaning of “radially-adjacent” (and, in guidewire 15 shown in FIG. 2A, coil 36 is, in fact, in direct physical contact with core 30 so as to be conductively coupled thereto).

More particularly, coil 36 is radially-adjacent to core 30 and circumferentially surrounds a first portion of the length of core 30. In turn, insulative jacket 38 is radially-adjacent coil 36 and circumferentially surrounds a second portion of the length of core 30 as well as a portion of the length of coil 36.

It should be understood that the first portion of the length of core 30 (e.g., the portion that is circumferentially surrounded by coil 36) and the second portion of the length of core 30 (e.g., the portion that is circumferentially surrounded by insulative jacket 38) may be the same or different and may be overlapping or non-overlapping. To illustrate, coil 36 may have proximal and distal termination points 40, 42 that are respectively located distally of the proximal and distal termination points 44, 46 of insulative jacket 38, such that the first and second portions of the length of core 30 are different and overlapping. In such a configuration, coil 36 can include an exposed distal segment 48 that extends beyond distal termination point 46 of insulative jacket 38, though it is also contemplated that coil 36 may instead be fully covered by insulative jacket 38. As described in further detail below, exposed distal segment 48 may work in conjunction with an energy delivery element to deliver penetrating energy to a tissue. Likewise, much of the length of core 30 will be circumferentially surrounded only by insulative jacket 38 and not also by coil 36.

In particular aspects of the disclosure, core 30 may have an overall length of about between about 150 cm (about 59 inches) and about 300 cm (about 118.1 inches), such as about 180 cm (about 71 inches) or about 230 cm (about 90.55 inches); coil 36 may have an overall length of between about 20 mm (about 0.79 inches) and about 500 mm (about 19.7 inches), such about 79 mm (about 3.1 inches); and insulative jacket 38 may have an overall length of between about 170 cm (about 67 inches) and about 190 cm (about 74.8 inches), such as about 178 cm (about 70 inches). Proximal termination point 44 of insulative jacket 38 may be between about 5 mm (about 0.20 inches) and about 30 mm (about 1.18 inches), such as about 13 mm (about 0.5 inches), from proximal end 32 of guidewire 15. This creates an exposed proximal segment 50 of core 30 having a corresponding length that may be conductively coupled to a power supply, for example, a radiofrequency (RF) energy generator capable of producing a power of up to about 50 W and a voltage of up to about 500 V, or an electrosurgical generator, such as the VIO® 300D electrosurgical system (Erbe USA, Inc.; Marietta, Georgia). Similarly, exposed distal segment 48 of coil 36 may be between about 0.10 mm (about 0.004 inches) and about 0.70 mm (about 0.027 inches), such as about 0.25 mm (about 0.010 inches), long.

As seen in FIG. 2A, core 30 can have a diameter that tapers distally along the length of core 30. The diameter of core 30 may be between about 0.5 mm (about 0.020 inches) to about 0.7 mm (about 0.028 inches), such as about 0.6 mm (about 0.024 inches), more proximally, tapering to a diameter of between about 0.1 mm (about 0.004 inches) and about 0.3 mm (about 0.012 inches), such as about 0.2 mm (about 0.008 inches), more distally. The tapering may occur over a relatively short length, such as between about 25 mm (about 1 inch) and about 500 mm (about 20 inches). The tapering may be linear, parabolic, or any other desired shape. A parabolic shape may advantageously provide improved handling of guidewire 15.

As will be familiar to those of ordinary skill in the art, coil 36 lends flexibility to guidewire 15. To this end, coil 36 will typically not be secured to core 30 along its entire length. Rather, coil 36 may be secured at its distal end to core 30, and may further be secured to core 30 at a relatively small number of additional points along its length, such as at its proximal end and its midpoint. Further, the coil can be either single-filar or multi-filar (e.g., 2 to 10 filar, such as 6 filar).

Coil 36 may include an electrically-conductive material. Coil 36 may include a heavy metal, such as tungsten, platinum, gold, iridium, palladium, and/or alloys thereof. Such materials may desirably enhance radiopacity of guidewire 15.

Various polymeric materials, including polytetrafluoroethylene (PTFE), polyether ether ketone (PEEK), various polyether block amides (e.g., PEBAX), polyimide (PI), polyolefin, and epoxy are suitable for use in insulative jacket 38. Nylon is also suitable for use in insulative jacket 38. Insulative jacket 38 may also include a coating, such as epoxy coatings (e.g., Electrobond® epoxy coating (Surface Solutions Group, LLC; Chicago, IL)), polyimide coating, ceramic coating, or alumina coating.

An energy delivery element 52 is positioned at distal end 34 of core 30. Energy delivery element 52 is configured and operable to deliver sufficient energy (e.g., ablative energy) to a tissue (e.g., the fossa ovalis) adjacent energy delivery element 52 to permit guidewire 15 to penetrate the tissue (“penetrating energy,” as discussed further below).

Energy delivery element 52 may be suitable for delivering radiofrequency (RF) energy to adjacent tissue to effect thermal ablation and/or irreversible electroporation (IRE) (also known as pulsed-field ablation (PFA)) of the tissue. To this end, energy delivery element 52 may be conductively coupled to core 30, which, as mentioned above, may in turn be conductively coupled to an RF energy generator and/or an electrosurgical generator.

Energy delivery element 52 may include one or more acoustic elements (e.g., ultrasound transducers).

Energy delivery element 52 can be capable of generating electromagnetic fields strong enough to cause enough damage to adjacent tissue to enable penetration by guidewire 15.

As one example, to raise the temperature of adjacent tissue beyond about 100 degrees C. and cause tissue evaporation around energy delivery element 52 to enable penetration by guidewire 15, the peak RF voltage at energy delivery element 52 can be up to about 300 V (or higher) and the RF power at energy delivery element 52 can be about 10 W or greater.

Various forms of energy delivery element 52 are contemplated. For instance, energy delivery element 52 may be a solder ball having a diameter of between about 1 mm (0.039 inches) and about 2 mm (0.079 inches), such as about 0.6 mm (about 0.025 inches) that is applied to distal end 34 of core 30.

Alternatively, energy delivery element 52 may be a machined element (e.g., a tip electrode) welded, adhesively bonded, brazed, or otherwise secured to distal end 34 of core 30.

Alternatively, energy delivery element 52 may be integrally formed with core 30 and coil 36, such as by shaping distal end 34 of core 30 into a desired shape by soldering or plasma welding.

Energy delivery element 52 may also include a conductive plating, such as of gold or platinum. Such a conductive plating may advantageously improve the performance of energy delivery element 52 for tissue penetration (e.g., transseptal crossing), as well as for measurement of intracardiac electrograms.

As shown in FIG. 2A, coil 36 may also be conductively-coupled to core 30 through direct conductive contact therebetween. Thus, ablative energy may be delivered to tissue not only through energy delivery element 52, but also through exposed distal segment 48 of coil 36. That is, exposed distal segment 48 of coil 36 can act in concert with energy delivery element 52 to deliver penetrating energy to a tissue as discussed in greater detail below.

A variation of guidewire 15 is shown in FIG. 2B. This variation of guidewire 15 interposes an optional insulative component 53 proximate the interface between energy delivery element 52 and insulative jacket 38.

As those of ordinary skill in the art will appreciate, the highest current density during operation of guidewire 15 will be at the interface between energy delivery element 52/coil 36 and insulative jacket 38 due to the large difference in electrical conductivity therebetween. This electrical concentration will result in higher temperatures that may thermally degrade insulative jacket 38.

Insulative component 53, however, shifts the area of highest current density, and thus highest temperature, from the distal end of insulative jacket 38 to the interface between energy delivery element 52 and insulative component 53. This, in turn, prolongs the useful life of guidewire 15 by minimizing the risk of thermal degradation in insulative jacket 38 during operation of guidewire 15.

Suitable materials for insulative component 53 include various coatings, such as Electrobond® epoxy coating, various ceramic coatings, alumina coatings, diamond-like carbon (DLC) coatings (e.g., CeraTough™-D (IBC Coatings Technologies, Ltd.; Lebanon, IN)), and polyimide coatings. These materials have higher melting points than the polymers, such as PTFE, PEEK, and/or PEBAX, that may be used for insulative jacket 38. For instance, PTFE has a melting point of about 260 degrees Celsius, while Electrobond® epoxy coating and certain DLC coatings have melting points of about 450 degrees Celsius. Thus, the use of insulative component 53 advantageously improves the thermal performance of guidewire 15.

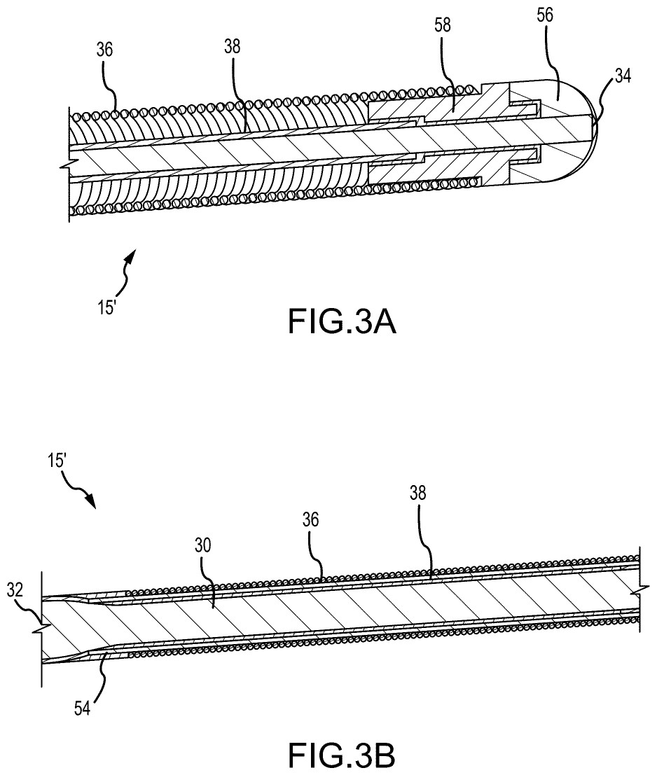

FIGS. 3A and 3B respectively illustrate a distal portion and a proximal portion of a guidewire 15′ according to a second embodiment of the disclosure. Like guidewire 15, guidewire 15′ includes an elongate core 30 having a proximal end and a distal end 34 (shown in FIG. 3A). As in the case of guidewire 15, core 30 of guidewire 15′ is electrically-conductive and may, for instance, be made of solid metal such as 304 stainless steel, Nitinol (nickel titanium alloy), or another suitable conductor.

Core 30 is circumferentially surrounded, as successive radially-adjacent components, by insulative jacket 38 and coil 36. In guidewire 15′ shown in FIGS. 3A and 3B, however, the arrangement of insulative jacket 38 and coil 36 is reversed from that of guidewire 15 shown in FIGS. 2A and 2B. That is, in guidewire 15′, insulative jacket 38 is radially-adjacent both core 30 and coil 36, while coil 36 is only radially-adjacent insulative jacket 38.

Coil 36 may have an overall length of about 79 mm (about 3.1 inches). As shown in FIG. 3B, coil 36 may be positioned such that it circumferentially surrounds the narrower-diameter portion (that is, the tapered portion) of core 30.

By reversing the arrangement of coil 36 and insulative jacket 38 in guidewire 15′ relative to that of guidewire 15, insulative jacket 38 electrically isolates coil 36 from core 30 along their respective lengths. Further, by positioning insulative jacket 38 radially-inward of coil 36, guidewire 15′ may have handling behavior more analogous to conventional guidewires (e.g., as used in extant electrophysiology procedures).

As mentioned above, insulative jacket 38 may be made of PTFE, PEEK, various polyether block amides (e.g., PEBAX), or another polymer material. Insulative jacket 38 may also include a coating, such as an Electrobond® epoxy coating, a polyimide coating, a perylene coating, a ceramic coating, or an alumina coating.

An additional insulative jacket 54, such as a length of polyimide tubing, may be provided to electrically isolate the proximal end of coil 36 from the wider-diameter, more proximal portion of core 30. As illustrated to good advantage in FIG. 3B, the diameter of coil 36 approximates the diameter of the more proximal portion (that is, the untapered portion) of core 30. Thus, insulative jacket 54 provides an electrically-insulative buffer between the proximal termination of coil 36 and the wider-diameter, untapered portion of core 30.

As a structure analogous to energy delivery element 52 shown in FIGS. 2A and 2B, a tip electrode 56 is conductively coupled to distal end 34 of core 30, such as by attaching tip electrode 56 to core 30 by welding, soldering, or electrically-conductive epoxy. To facilitate the foregoing, insulative jacket 38 and coil 36 may both terminate before distal end 34 of core 30, thus exposing a distal portion of core 30 for conductive connection to tip electrode 56. Tip electrode 56 may be made of any suitable conductor, including, without limitation, platinum, gold, or stainless steel. Alternatively or additionally, tip electrode 56 may include a conductive plating, such as of platinum or gold. As mentioned above, the conductive plating may improve the conductive properties of tip electrode 56 for delivering penetrating energy as well as for measurement of intracardiac electrograms.

To electrically isolate coil 36 from tip electrode 56, an insulative bushing 58 may be positioned between tip electrode 56 and coil 36. The distal end of bushing 58 may be attached to the tip electrode 56 and the proximal end of bushing 58 may be attached to a distal portion of coil 36; bushing 58 may also be secured (e.g., via adhesive) to core 30. Bushing 58 may be made of polyether-ether ketone (PEEK), nylon, or another suitable insulative material (including the materials described above in connection with insulative component 53), or polyimide tubing filled with epoxy (e.g., Epo-Tek® 353 heat cure epoxy (Epoxy Technology; Billerica, MA)).

As in the case of guidewire 15, the proximal termination point of insulative jacket 38 in guidewire 15′ may be about 13 mm (about 0.5 inches) from the proximal end of guidewire 15′ to create an exposed proximal segment. The exposed proximal segment may be conductively coupled to power supply, such as a radiofrequency (RF) energy generator or electrosurgical generator, as discussed above.

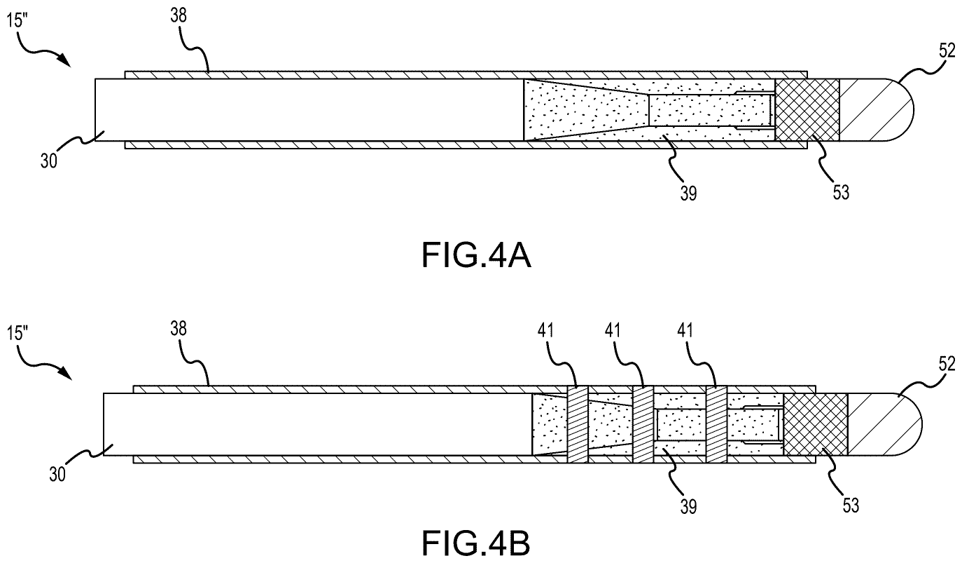

FIG. 4A illustrates a guidewire 15″ according to further aspects of the disclosure. Guidewire 15″ is generally analogous to the variation of guidewire 15 shown in FIG. 2B; certain aspects of guidewire 15″ will be described in further detail below.

Like guidewires 15 and 15′, guidewire 15″ includes an elongate core 30 having a proximal end and a distal end. As discussed above, core 30 of guidewire 15″ is electrically-conductive and may, for instance, be made of solid metal such as 304 stainless steel, Nitinol (nickel titanium alloy), or another suitable conductor.

Core 30 of guidewire 15″ is circumferentially surrounded by insulative jacket 38.

Rather than including a circumferentially-surrounding coil, however, guidewire 15″ includes a filler material 39 between core 30 and insulative jacket 38.

Filler material 39 may include a polymeric material, such as PEBAX of varying durometer, and may be incorporated by reflow bonding. Alternatively, filler material 39 may be incorporated via dip coating a suitable material, such as pellethane. It is also contemplated that filler material 39 may be loaded with one or more heavy metals, such as tungsten or gold, to increase radiopacity of the distal portion of guidewire 15″.

As shown in FIG. 4B, the distal portion of guidewire 15″ may also include one or more electrodes 41. As those of ordinary skill in the art will recognize, electrodes 41 may be used to localize and/or visualize guidewire 15″ within a system 70 (e.g., an electroanatomical mapping system) as mentioned below.

Those of ordinary skill in the art will be familiar with the construction of elongate medical devices that include electrodes (e.g., ring electrodes) within their distal portions, such that a detailed explanation of electrodes 41 and their respective electrical connections (e.g., to system 70 discussed below) need not be provided herein. As one example, however, it is contemplated that electrodes 41 may be discrete platinum/iridium electrodes connected to corresponding signal wires that travel along the length of guidewire 15″ to a suitable connector at the proximal end thereof. The signal wires may be positioned between core 30 and insulative jacket 38 (and an insulative coating, such as PTFE, may be applied to the exterior surface of core 30 to improve electrical insulation between core 30 and the signal wires) or may be routed through a lumen within core 30 (e.g., core 30 may be hollow). Electrode pads (not shown) may be located on the outer surface of guidewire 15″, insulated from the guidewire 15″, and provide for connection between the electrodes 41 and signal wires.

As another example, electrodes 41 and/or their corresponding connectors may be included on a flexible electronic circuit. The flexible electronic circuit may be wrapped around the core 30. The flexible electronic circuit can include insulative materials. Various suitable configurations of electrodes and connectors that utilize flexible electronic circuits are described in U.S. provisional application No. 63/702,909 , which is hereby incorporated by reference as though fully set forth herein.

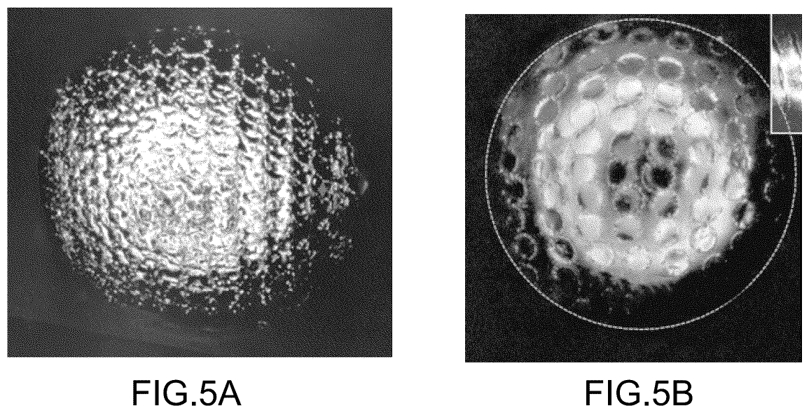

It is also contemplated that energy delivery element 52 (as shown in FIGS. 2A and 2B), tip electrode 56 (as shown in FIGS. 3A, 4A, and 4B), and/or coil 36 can include a roughened texture as shown in FIGS. 5A and 5B. These roughened texture surfaces increase the surface area of energy delivery element 52, tip electrode 56, and/or coil 36, in turn improving the performance of guidewire 15, 15′, 15″ for transseptal puncture and offering increased visibility of guidewire 15, 15′, 15″ under acoustic imaging (e.g., intracardiac electrocardiogramar transesophageal echocardiography (TEE)) through enhanced echogenicity of guidewire 15, 15′, 15″ resulting from omnidirectional ultrasound reflection from delivery element 52, tip electrode 56, and/or coil 36.

The roughened texture surface may be achieved by bead blasting, laser roughening, chemical etching, electrochemical machining (ECM), spray coating, plasma spraying, sputtering, or another technique suitable for increasing the microscopic surface area of energy delivery element 52, tip electrode 56, and/or coil 36. Laser roughening can be used to obtain an overlapping roughened texture (FIG. 5A), or a more regular and repeating pattern, for example a regularized distribution of dimples (FIG. 5B), as might be seen on a golf ball. Laser roughening can achieve the roughened surface texture in a relatively short timescale, for example, between about 0.1 to about 2 seconds, and preferably about 0.5 seconds. Other roughened surface textures and patterns are envisaged.

The overall surface roughness (average roughness, Ra) may be in the range of about 1 micron to about 5 microns, more particularly between about 2 microns and about 4 microns, and even more particularly about 3 microns. The dimple diameter may be between about 1 micron and about 30 μm, more particularly between about 5 microns and about 25 μm, and even more particularly between about 10 microns and about 20 microns.

The roughened texture surface enhances engagement between energy delivery element 52, tip electrode 56, and/or coil 36, on the one hand, and the adjacent tissue, on the other hand by inhibiting relative sliding therebetween.

It is contemplated that the surface roughening and electrode plating may be applied simultaneously. Alternatively, the surface roughening and electrode plating may be performed serially in any order. That is, the conductive plating may comprise a roughened texture surface as described above.

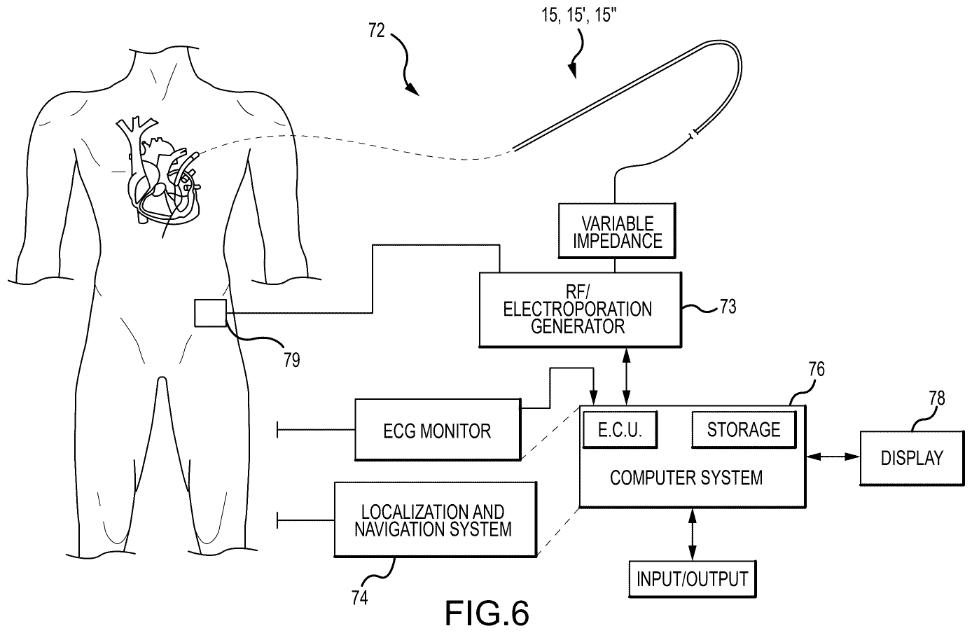

FIG. 6 is a diagrammatic and block diagram view of a system 70 that may be utilized in accordance with aspects of the instant disclosure. FIG. 6 schematically illustrates guidewire 15, 15′, 15″ as connected to electronics 72 within system 70. As those of ordinary skill in the art will appreciate, and as shown in FIG. 6, electronics 72 may include an energy generator 73, an electroanatomical mapping system 74, a computer system 76, a display 78, and the like. Insofar as the components of system 70 will be familiar to those of ordinary skill in the art, they need not be described in detail herein.

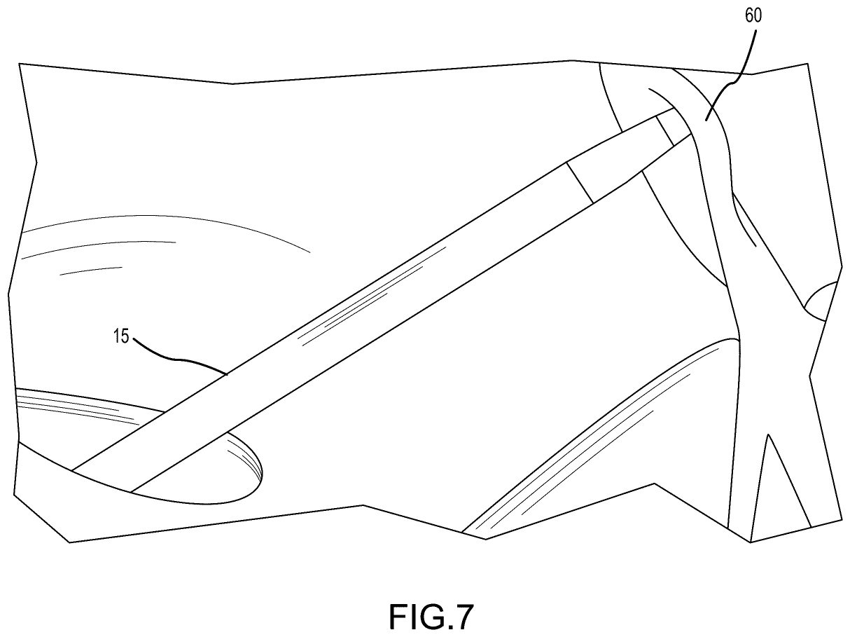

In use, and as illustrated in FIG. 7, guidewire 15, 15′, 15″ is advanced through a subject's vasculature, optionally with the aid of introducer 12 and/or dilator 14 (not shown in FIG. 7 for the sake of clarity), and into the subject's right atrium. Distal end 34 of guidewire 15, 15′, 15″ is used to locate the fossa ovalis 60 in a manner known to those of ordinary skill in the art (though, as noted above, energy delivery element 52, tip electrode 56, and/or additional electrodes 41 may simplify this aspect of the procedure insofar as, unlike extant transseptal catheterization apparatus, it allows guidewire 15, 15′, 15″ to be localized and visualized via an electroanatomical mapping system, fluoroscopy system, and/or acoustic imaging system, and may therefore enhance a practitioner's ability to ensure guidewire 15, 15′, 15″ is adjacent the fossa ovalis).

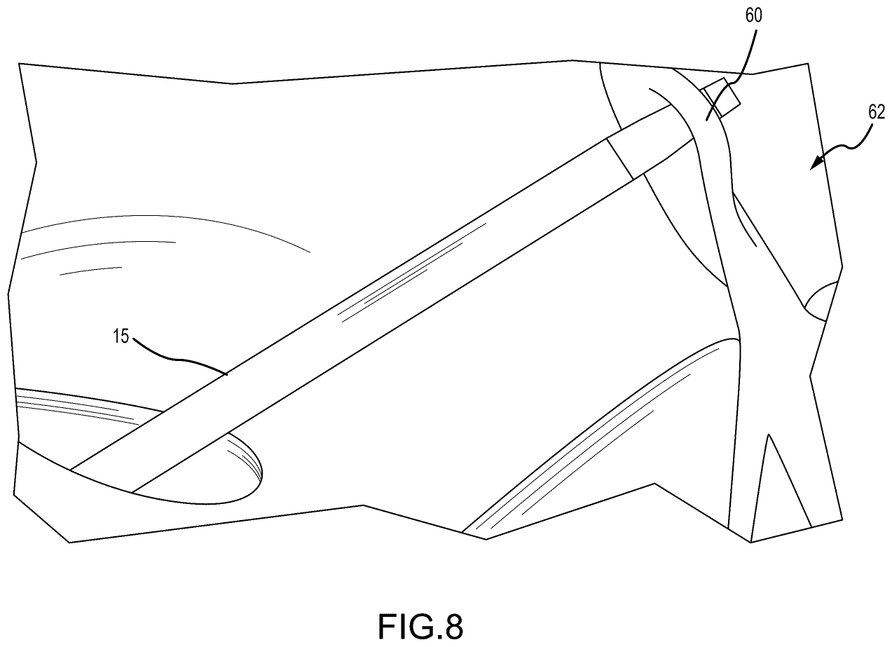

Once tenting of fossa ovalis 60 is observed, energy delivery element 52/tip electrode 56 can be activated to deliver sufficient energy (e.g., via energy generator 73) to allow guidewire 15, 15′, 15″ (and, optionally, introducer 12 and/or dilator 14) to penetrate fossa ovalis 60 and cross into the left atrium 62 as shown in FIG. 8 (referred to herein as “penetrating energy”).

As briefly mentioned above, various modalities for delivery of penetrating energy are contemplated. For example, energy delivery element 52/tip electrode 56 may be a radiofrequency (RF) ablation element, and energy generator 73 may deliver sufficient RF energy to penetrate fossa ovalis 60 and cross into the left atrium 62.

Alternatively, energy delivery element 52/tip electrode 56 may be configured to deliver irreversible electroporation therapy (IRE) (also known as pulsed field ablation (PFA) therapy), and energy generator 73 may deliver energy pulses to penetrate fossa ovalis 60 and cross into left atrium 62. Where only a single energy delivery element 52/tip electrode 56 is present on guidewire 15, 15′, 15″ guidewire 15, 15′, 15″ can serve as a monopolar IRE/PFA probe. Alternatively, if guidewire 15, 15′, 15″ includes two or more energy delivery elements, it can be used to as a bipolar IRE/PFA or RF probe.

Energy delivery element 52 may include one or more acoustic elements (e.g., ultrasound transducers), and energy generator 73 may be an acoustic generator.

Energy generator 73 can power energy delivery element 52/tip electrode 56 to generate an electromagnetic field strong enough to cause enough damage to fossa ovalis 60 to enable penetration by guidewire 15, 15′, 15″.

The penetrating energy can be between about 5 W and about 20 W of energy applied over a time interval of about 0.11 second to about 10 seconds. In other embodiments of the disclosure, the penetrating energy can be about 429V peak 30 W maximum applied over a time interval of about 0.25 seconds to about 2 seconds or about 520 V peak 30 W maximum in multiple bursts of about 40 ms in length separated by about 80 ms intervals. It should be understood, however, that other power levels and/or time intervals are regarded as within the spirit and scope of the instant disclosure.

Energy delivery element 52/tip electrode 56 may operate as a source electrode for the penetrating energy and a patch electrode 79 may operate as the sink for the penetrating energy.

After guidewire 15, 15′, 15″ has crossed into left atrium 62, dilator 14 and introducer 12 may be advanced into left atrium 62 over guidewire 15. Next, dilator 14 and guidewire 15, 15′, 15″ may be removed, leaving introducer 12 resident in left atrium 62. Any desirable diagnostic or therapeutic device (e.g., an electrophysiology mapping catheter, an ablation catheter, or the like) can be advanced through introducer 12 into left atrium 62 according to methods that will be familiar to the ordinarily-skilled artisan.

Alternatively, dilator 14 and introducer 12 may be withdrawn, leaving guidewire 15, 15′, 15″ resident in left atrium 62. Any desirable diagnostic or therapeutic device (e.g., an electrophysiology mapping catheter, an ablation catheter, or the like) may then be advanced over guidewire 15, 15′, 15″ into left atrium 62 according to methods that will be familiar to the ordinarily-skilled artisan.

In any event, the teachings herein simplify transseptal crossing procedures by reducing device exchanges during the procedure.

Although several embodiments have been described above with a certain degree of particularity, those skilled in the art could make numerous alterations to the disclosed embodiments without departing from the spirit or scope of this invention.

For example, although FIGS. 2A, 2B, 3A, and 3B depict only a single coil 36, additional coils, which may be respectively arranged along the length of core 30 and/or disposed concentrically around core 30 and each other, are contemplated. Such additional coils may provide enhanced support and maneuverability for guidewire 15, 15′.

Likewise, although guidewires 15, 15′, 15″ are shown as having a generally straight tip in FIGS. 2A, 2B, 3A, 3B, 4A, and 4B other configurations (e.g., J-curve/shepherd's hook, Rosen, pigtail, and the like) are regarded as within the scope of the present disclosure.

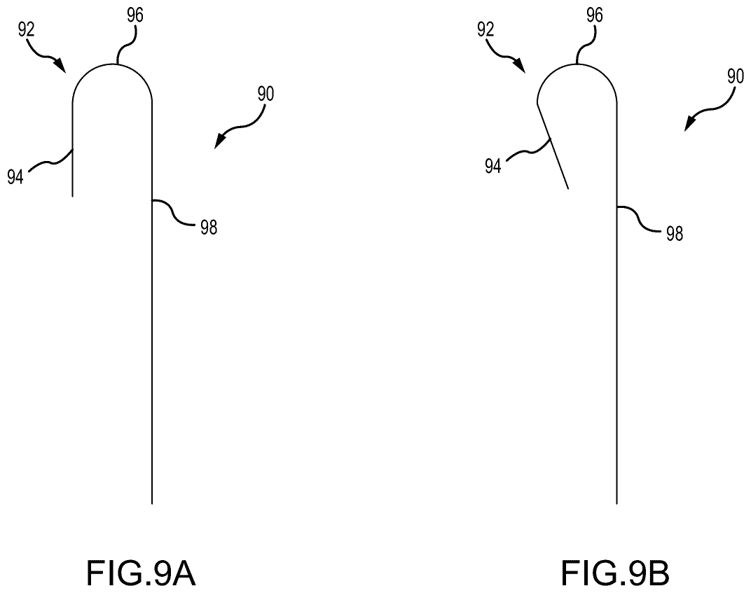

For instance, FIGS. 9A and 9B are illustrative curvatures of a transseptal guidewire 90 with a J-curve or shepherd's hook tip configuration. In FIG. 9A, the tip 92 includes a straight portion 94 and a curved portion 96. Curved portion 96 may have an arc length of about 180 degrees and a radius of curvature of between about 2 mm and about 4 mm, such as about 3 mm. Straight portion 94 may have a length of between about 5 mm and about 9 mm, such as about 7 mm.

As shown in FIG. 9B, straight portion 94 need not be substantially parallel to the more proximal portion 98 of guidewire 90. For example, straight portion 94 may deviate from parallel to proximal portion 98 of guidewire 90 by between about 15 degrees and about 25 degrees, such as about 20 degrees.

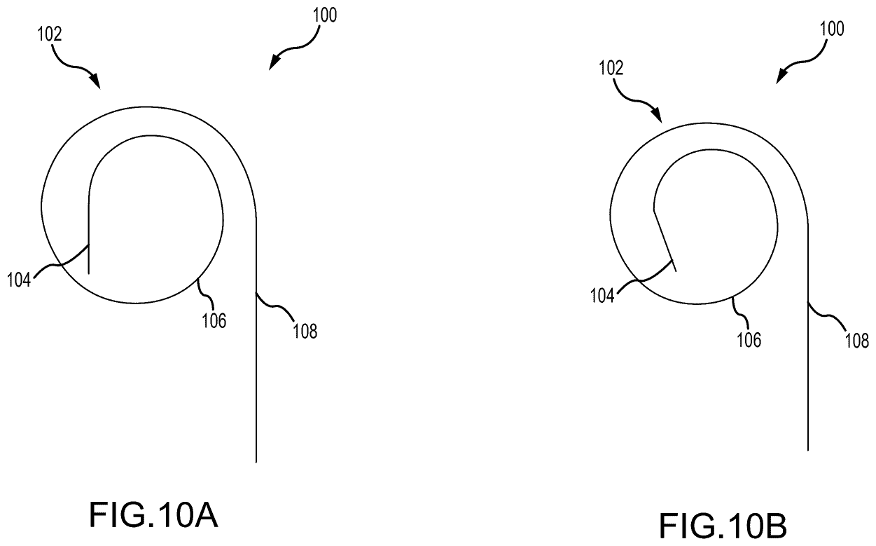

Similarly, FIGS. 10A and 10B are illustrative curvatures of a transseptal guidewire 100 with a pigtail tip configuration. In FIG. 10A, the tip 102 has a straight portion 104 and a spiral portion 106. Spiral portion 106 may start at a diameter of between about 18 mm and about 25 mm, such as about 21 mm and gradually spiral inward to a diameter of between about 12 mm and about 16 mm, such as about 14 mm. Straight portion 104 may have a length of between about 4 mm and about 10 mm, such as about 6 mm.

As shown in FIG. 10B, straight portion 104 need not be substantially parallel to the more proximal portion 108 of guidewire 100. For example, straight portion 104 may deviate from parallel to proximal portion 108 of guidewire 100 by between about 15 degrees and about 25 degrees, such as about 20 degrees.

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

It is intended that all matter contained in the above description or shown in the accompanying drawings shall be interpreted as illustrative only and not limiting. Changes in detail or structure may be made without departing from the spirit of the invention as defined in the appended claims.

The invention is defined in the appended claims. A non-exhaustive list of aspects of the invention set out in the numbered clauses is useful for understanding the invention. The following relate to numbered clauses of the invention:
Clause 1: An apparatus for transseptal catheterization, comprising:
   an electrically-conductive core having a proximal end, a distal end, and a length extending from the proximal end to the distal end;
   an energy delivery element positioned at the distal end of the core;
   a coil circumferentially surrounding a first portion of the length of the core; and
   an insulative jacket circumferentially surrounding a second portion of the length of the core.
Clause 2: The apparatus according to clause 1, wherein the coil is radially adjacent the core and the insulative jacket circumferentially surrounds a portion of a length of the coil.
Clause 3: The apparatus according to clause 2, wherein the coil comprises an exposed segment extending beyond a distal end of the insulative jacket.
Clause 4: The apparatus according to clause 3, wherein the exposed segment of the coil has a length between 0.10 mm and 0.70 mm, optionally between 0.20 mm (0.008 inches) and 0.30 mm (0.012 inches).
Clause 5: The apparatus according to any preceding clause, wherein the coil is conductively coupled to the core.
Clause 6: The apparatus according to clause 5, wherein the coil is in direct conductive contact with the core.
Clause 7: The apparatus according to clauses 1 to 4, wherein the coil is electrically isolated from the core and the energy delivery element.
Clause 8: The apparatus according to clause 7, wherein the insulative jacket is radially adjacent the core and the coil circumferentially surrounds a portion of a length of the insulative jacket.
Clause 9: The apparatus according to clause 7 or 8, wherein the energy delivery element comprises a tip electrode conductively coupled to the distal end of the core.
Clause 10: The apparatus according to clause 9, further comprising an insulative bushing positioned between a proximal end of the tip electrode and a distal end of the coil to electrically isolate the tip electrode from the coil.
Clause 11: The apparatus according to any preceding clause, wherein the core has a diameter, and wherein the diameter of the core tapers moving distally along the length of the core.
Clause 12: The apparatus according to clause 11, wherein the diameter of the core at the proximal end is between 0.25 mm (0.01 inches) and 1.27 mm (0.05 inches), between 0.38 mm (0.015 inches) and 1.02 mm (0.04 inches), or between 0.51 mm (0.02 inches) and 0.76 mm (0.03 inches).
Clause 13: The apparatus according to clause 11 or 12, wherein the diameter of the core at the distal end is between 0.051 mm (0.002 inches) and 0.25 mm (0.01 inches), between 0.10 mm (0.004 inches) and 0.20 mm (0.008 inches), or between 0.15 mm (0.006 inches) and 0.18 mm (0.007 inches).
Clause 14: The apparatus according to clauses 11 to 13, wherein the diameter of the core tapers over a length along the core of between 12.7 mm (0.5 inches) and 508 mm (20 inches), between 50.8 mm (2 inches) and 305 mm (12 inches), or between 152 mm (6 inches) and 254 mm (10 inches).
Clause 15: The apparatus according to any preceding clause, wherein the core comprises a metal or metal alloy.
Clause 16: The apparatus according to clause 15, wherein the metal or metal alloy comprises one or more of stainless steel, platinum, platinum-iridium and Nitinol (nickel titanium alloy).
Clause 17: The apparatus according to any preceding clause, wherein the coil comprises a heavy metal.
Clause 18: The apparatus according to clause 17, wherein the heavy metal comprises one or more of tungsten, platinum, and gold, or alloys thereof.
Clause 19: The apparatus according to any preceding clause, wherein the insulative jacket comprises a non-conductive polymer.
Clause 20: The apparatus according to clause 19, wherein the non-conductive polymer comprises one or more of polytetrafluoroethylene, polyether ether ketone, nylon, polyimide, epoxy, polyolefin, or a polyether block amide.
Clause 21: The apparatus according to any preceding clause, wherein the energy delivery element comprises one or more of platinum, platinum iridium, palladium, gold, and stainless steel.
Clause 22: The apparatus according to any preceding clause, wherein at least one of an exterior surface of the energy delivery element and an exterior surface of the coil comprises a roughened texture.
Clause 23: The apparatus according to clause 22, wherein the roughened texture comprises a distribution of dimples.
Clause 24: The apparatus according to clause 22 or 23, wherein the roughened texture is fabricated using at least one of laser ablation, chemical etching, electrochemical machining, spray coating, plasma spraying, or sputtering.
Clause 25: The apparatus according to clauses 22 to 24, wherein the roughened texture has an average roughness (Ra) between 1 micron and 5 microns, between 2 microns and 4 microns, or 3 microns.
Clause 26: The apparatus according to any preceding clause, wherein the energy delivery element comprises a conductive plating.
Clause 27: The apparatus according to clause 26, wherein the conductive plating comprises at least one of platinum and gold.
Clause 28: The apparatus according to any preceding clause, wherein the distal portion of the coil comprises a conductive plating.
Clause 29: The apparatus according to clause 28, wherein the conductive plating comprises one or more of platinum and gold.
Clause 30 The apparatus according to any preceding clause, wherein a proximal segment of the core is exposed beyond a proximal end of the coil and a proximal end of the insulative jacket.
Clause 31: The apparatus according to clause 30, wherein the exposed proximal segment of the core is configured to be conductively connected to a power supply.
Clause 32: The apparatus according to any preceding clause, further comprising a second coil radially adjacent the core and the coil.
Clause 33: A guidewire for radiofrequency transseptal catheterization, comprising:
   an electrically-conductive core having a proximal end, a distal end, and a length extending from the proximal end to the distal end;
   a coil circumferentially surrounding a portion of the length of the core, wherein the coil is conductively coupled to the core; and
   an insulative jacket circumferentially surrounding a portion of a length of the coil, wherein a distal segment of the coil is exposed beyond a distal end of the insulative jacket to act as a conductive element to deliver sufficient energy to a tissue adjacent the energy delivery element to permit the guidewire to penetrate the tissue.
Clause 34: The guidewire according to clause 33, wherein a proximal segment of the core is exposed beyond a proximal end of the coil and a proximal end of the insulative jacket and is configured to be conductively connected to a radiofrequency generator.
Clause 35: The guidewire according to clause 33 or 34, further comprising a tip electrode conductively coupled to at least one of the distal end of the core and the exposed distal segment of the coil.
Clause 36: A guidewire for radiofrequency transseptal catheterization, comprising:
   an electrically-conductive core having a proximal end, a distal end, and a length extending from the proximal end to the distal end;
   a tip electrode coupled to the distal end of the core and configured to deliver sufficient energy to a tissue adjacent the tip electrode to permit the guidewire to penetrate the tissue;
   an insulative jacket circumferentially surrounding a portion of the length of the core; and
   a coil circumferentially surrounding a portion of a length of the insulative jacket, wherein the coil is electrically isolated from the core and the tip electrode.
Clause 37: The guidewire according to clause 36, further comprising an insulative bushing positioned between a proximal end of the tip electrode and a distal end of the coil.
Clause 38: A method of manufacturing an apparatus for transseptal catheterization, comprising:
   forming an electrically-conductive core having a proximal end, a distal end, and a length extending from the proximal end to the distal end;
   positioning an energy delivery element at the distal end of the core;
   forming a coil to circumferentially surround a first portion of the length of the core; and
   forming an insulative jacket to circumferentially surround a second portion of the length of the core.
Clause 39: The method according to clause 38, wherein the coil is radially adjacent the core and forming the insulative jacket to circumferentially surround the second portion of the length of the core further comprises forming the insulative jacket to circumferentially surround a portion of a length of the coil.
Clause 40: The method according to clause 38, wherein the insulative jacket is radially adjacent the core and forming the coil to circumferentially surround the first portion of the length of the core further comprises forming the coil to circumferentially surround a portion of a length of the insulative jacket.
Clause 41: The method according to clauses 38 to 40, wherein positioning the energy delivery element at the distal end of the core comprises securing a tip electrode to the distal end of the core.
Clause 42: The method according to clauses 38 to 40, wherein positioning the energy delivery element at the distal end of the core comprises forming a solder ball on the distal end of the core.
Clause 43: The method according to clauses 38 to 42, further comprising forming a roughened texture surface on at least one of the coil and the energy delivery element.
Clause 44: The method according to clause 43, wherein forming the roughened texture surface on the at least one of the coil and the energy delivery element comprises laser roughening the at least one of the coil and the energy delivery element.
Clause 45: An apparatus for transseptal catheterization, comprising:
   an electrically-conductive core having a proximal end, a distal end, and a length extending from the proximal end to the distal end;
   an energy delivery element positioned at the distal end of the core;
   an insulative filler circumferentially surrounding a first portion of the length of the core; and
   an insulative jacket circumferentially surrounding a second portion of the length of the core.

What is claimed is:

1. An apparatus for transseptal catheterization, comprising:
an electrically-conductive core having a proximal end, a distal end, and a length extending from the proximal end to the distal end;
an energy delivery element positioned at the distal end of the core;
a coil circumferentially surrounding a first portion of the length of the core; and
an insulative jacket circumferentially surrounding a second portion of the length of the core.

2. The apparatus according to claim 1, wherein the coil is radially adjacent the core and the insulative jacket circumferentially surrounds a portion of a length of the coil.

3. The apparatus according to claim 2, wherein the coil is conductively coupled to the core.

4. The apparatus according to claim 3, wherein the coil is in direct conductive contact with the core.

5. The apparatus according to claim 1, wherein the core has a diameter, and wherein the diameter of the core tapers moving distally along the length of the core.

6. The apparatus according to claim 1, wherein the core comprises a metal or metal alloy.

7. The apparatus according to claim 6, wherein the metal or metal alloy comprises one or more of stainless steel, platinum, platinum-iridium, and Nitinol (nickel titanium alloy).

8. The apparatus according to claim 1, wherein the coil comprises a heavy metal.

9. The apparatus according to claim 8, wherein the heavy metal comprises one or more of tungsten, platinum, and gold, or alloys thereof.

10. The apparatus according to claim 1, wherein the insulative jacket comprises a non-conductive polymer.

11. The apparatus according to claim 10, wherein the non-conductive polymer comprises one or more of polytetrafluoroethylene, polyether ether ketone, nylon, polyimide, epoxy, polyolefin, or a polyether block amide.

12. The apparatus according to claim 1, wherein the energy delivery element comprises one or more of platinum, platinum iridium, palladium, gold, and stainless steel.

13. The apparatus according to claim 1, wherein a proximal segment of the core is exposed beyond a proximal end of the coil and a proximal end of the insulative jacket.

14. The apparatus according to claim 13, wherein the exposed proximal segment of the core is configured to be conductively connected to a power supply.

15. A method of manufacturing an apparatus for transseptal catheterization, comprising:
forming an electrically-conductive core having a proximal end, a distal end, and a length extending from the proximal end to the distal end;
positioning an energy delivery element at the distal end of the core;
forming a coil to circumferentially surround a first portion of the length of the core; and
forming an insulative jacket to circumferentially surround a second portion of the length of the core.

16. The method according to claim 15, wherein the coil is radially adjacent the core and forming the insulative jacket to circumferentially surround the second portion of the length of the core further comprises forming the insulative jacket to circumferentially surround a portion of a length of the coil.

17. The method according to claim 15, wherein positioning the energy delivery element at the distal end of the core comprises securing a tip electrode to the distal end of the core.

18. The method according to claim 15, further comprising forming a roughened texture surface on at least one of the coil and the energy delivery element.

19. The method according to claim 18, wherein forming the roughened texture surface on the at least one of the coil and the energy delivery element comprises laser roughening the at least one of the coil and the energy delivery element.

20. An apparatus for transseptal catheterization, comprising:
an electrically-conductive core having a proximal end, a distal end, and a length extending from the proximal end to the distal end;
an energy delivery element positioned at the distal end of the core;
an insulative filler circumferentially surrounding a first portion of the length of the core; and
an insulative jacket circumferentially surrounding a second portion of the length of the core.
